(19) United States
(12) Patent Application Publication
(10) Pub. No.: US 2025/0128023 A1
(43) Pub. Date: Apr. 24, 2025

(54) SCAFFOLD INTRODUCER

(71) Applicant: iMacular Regeneration LLC, Rochester, MN (US)

(72) Inventors: Timothy W. Olsen, Rochester, MN (US); Mikhail Borisovich Tikh, St. Louis Park, MN (US); Jeffrey Robert Lind, Bloomington, MN (US); Antonio Joao Ubach, Tucson, AZ (US); John Christian Love, San Diego, CA (US)

(21) Appl. No.: 18/382,801

(22) Filed: Oct. 23, 2023

(51) Int. Cl. A61M 25/01

(52) U.S. Cl. CPC A61M 25/0102

(57) ABSTRACT

A device includes a deployment channel having a distal port and a proximal end disposed along a longitudinal axis. The device includes an actuator coupled to the deployment channel. The actuator is coupled to a pushrod disposed within a pushrod channel and aligned with the deployment channel. The pushrod is axially repositionable between a retract position and an eject position. A position of the pushrod relative to the pushrod channel is determined by the actuator.

### TECHNICAL FIELD

This document pertains generally, but not by way of limitation, to technology for performing a surgical procedure.

### BACKGROUND

Examples of technology for storing and dispensing articles include an ammunition magazine in the firearms industry and a PEZ dispenser in the candy industry. These examples are unsuitable for use in the medical field.

Systems and devices configured for dispensing articles in the medical field include the following:
U.S. Pat. No. 10,039,536 refers to an implantable medical device deployment system;
U.S. Pat. No. 10,966,850 refers to an implantable medical device constraint and deployment apparatus; and
US 2008/0188928 refers to a medical device delivery sheath.

### SUMMARY

An example of the present subject matter is configured to deliver, or convey, an implantable medical device to a surgical site and configured to encase the device prior to implantation. The medical device can include a scaffold for carrying a membrane useful for treatment of a condition known as macular degeneration.

In one example, the device remains in a substantially planar configuration when received in a channel, when ejected from the channel, and when implanted.

In one example, the device remains in a substantially planar configuration when received in the channel, and is manipulated into a curled configuration upon ejection from the channel and thereafter, the device reverts to a planar configuration prior to implantation.

In one example, the delivered object emerges from the device free of any rotation about an axis. In this manner, the object moves along a deployment channel, from a proximal end to a distal end, without rotating.

The present inventors have recognized, among other things, that a problem to be solved can include providing a device, a system, and a method for reliable and controlled delivery of a tissue support structure that protects the supported tissue from damage or contaminants in a surgical procedure for the eye. The present subject matter can help provide a solution to this problem, such as by providing a deployment channel and a distal port as described herein.

Aspect 1 can include or use subject matter (such as an apparatus, a system, a device, a method, a means for performing acts, or a device readable medium including instructions that, when performed by the device, can cause the device to perform acts, or an article of manufacture), such as can include or use a deployment channel having a distal port and a proximal end disposed along a longitudinal axis; and an actuator coupled to the deployment channel and coupled to a pushrod disposed within a pushrod channel and aligned with the deployment channel, the pushrod axially repositionable between a retract position and an eject position, wherein a position of the pushrod relative to the pushrod channel is determined by the actuator.

Aspect 2 can include or use, or can optionally be combined with the subject matter of Aspect 1, to optionally include or use wherein the pushrod includes an engagement pin aligned transverse to the deployment channel.

Aspect 3 can include or use, or can optionally be combined with the subject matter of one or any combination of Aspects 1 or 2 to optionally include or use wherein the deployment channel is tubular.

Aspect 4 can include or use, or can optionally be combined with the subject matter of one or any combination of Aspects 1 through 3 to optionally include or use wherein a cross section of the deployment channel is substantially rectangular.

Aspect 5 can include or use, or can optionally be combined with the subject matter of one or any combination of Aspects 1 through 4 to optionally include or use wherein the distal port includes an elongate aperture.

Aspect 6 can include or use, or can optionally be combined with the subject matter of one or any combination of Aspects 1 through 5 to optionally include or use wherein the actuator is slidably coupled to the deployment channel.

Aspect 7 can include or use, or can optionally be combined with the subject matter of one or any combination of Aspects 1 through 6 to optionally include or use further including a detent for positioning the pushrod relative to the pushrod channel.

Aspect 8 can include or use, or can optionally be combined with the subject matter of one or any combination of Aspects 1 through 7 to optionally include or use wherein the actuator includes a syringe plunger.

Aspect 9 can include or use, or can optionally be combined with the subject matter of one or any combination of Aspects 1 through 8 to optionally include or use wherein the deployment channel includes a surface inclined relative to the longitudinal axis.

Aspect 10 can include or use, or can optionally be combined with the subject matter of one or any combination of Aspect 9 to optionally include or use wherein the surface is disposed proximate the distal port.

Aspect 11 can include or use, or can optionally be combined with the subject matter of one or any combination of Aspects 1 through 10 to optionally include or use wherein the deployment channel includes a curl forming die.

Aspect 12 can include or use, or can optionally be combined with the subject matter of one or any combination of Aspects 1 through 11 to optionally include or use wherein the pushrod includes a round section.

Aspect 13 can include or use, or can optionally be combined with the subject matter of one or any combination of Aspects 1 through 12 to optionally include or use wherein the pushrod includes a shoulder at a selected axial position.

Aspect 14 can include or use, or can optionally be combined with the subject matter of one or any combination of Aspect 13 to optionally include or use wherein the shoulder is configured to exert a thrust force aligned with the longitudinal axis.

Aspect 15 can include or use, or can optionally be combined with the subject matter of one or any combination of Aspect 13 to optionally include or use wherein the deployment channel is configured to clear an end of the pushrod and wherein the shoulder is configured to exert a thrust.

Aspect 16 can include or use, or can optionally be combined with the subject matter of one or any combination of Aspect 15 to optionally include or use wherein the pushrod includes a relief disposed proximate the end.

Aspect 17 can include or use, or can optionally be combined with the subject matter of one or any combination of Aspects 1 through 16 to optionally include or use wherein the distal port includes a cannula.

Aspect 18 can include or use, or can optionally be combined with the subject matter of one or any combination of Aspect 17 to optionally include or use wherein the cannula includes an angle cut end.

Aspect 19 can include or use, or can optionally be combined with the subject matter of one or any combination of Aspect 17 to optionally include or use wherein the cannula includes a cutting edge.

Aspect 20 can include or use, or can optionally be combined with the subject matter of one or any combination of Aspects 1 through 19 to optionally include or use including a lock configured to immobilize the pushrod relative to the pushrod channel.

Aspect 21 can include or use, or can optionally be combined with the subject matter of one or any combination of Aspect 20 to optionally include or use wherein the lock includes an actuator saddle.

Aspect 22 can include or use, or can optionally be combined with the subject matter of one or any combination of Aspects 1 through 21 to optionally include or use wherein the deployment channel has a tapered wall disposed proximate the distal port.

Aspect 23 can include or use, or can optionally be combined with the subject matter of one or any combination of Aspect 22 to optionally include or use wherein the tapered wall includes a substantially circular cross section.

Aspect 24 can include or use subject matter (such as an apparatus, a system, a device, a method, a means for performing acts, or a device readable medium including instructions that, when performed by the device, can cause the device to perform acts, or an article of manufacture), such as can include or use a channel having an interior configured to receive a disk-shaped object and having a distal port for deploying the object; a pushrod configured for movement between a first axial position distal to the channel and a second axial position proximate to the channel, wherein an end of the pushrod is configured to exert a thrust force on the object and propel the object along an axis between the interior and the distal port; and an actuator coupled to the pushrod.

Aspect 25 can include or use, or can optionally be combined with the subject matter of Aspect 24, to optionally include or use wherein the channel includes a wall at an angle relative to the axis.

Aspect 26 can include or use, or can optionally be combined with the subject matter of one or any combination of Aspects 24 or 25 to optionally include or use wherein the interior is configured to deform the object with passage along the axis.

Aspect 27 can include or use, or can optionally be combined with the subject matter of one or any combination of Aspects 24 through 26 to optionally include or use wherein the distal port includes a cannula.

Aspect 28 can include or use, or can optionally be combined with the subject matter of one or any combination of Aspects 24 through 27 to optionally include or use wherein the cannula includes a cutting edge.

Aspect 29 can include or use subject matter (such as an apparatus, a system, a device, a method, a means for performing acts, or a device readable medium including instructions that, when performed by the device, can cause the device to perform acts, or an article of manufacture), such as can include or use a deployment channel configured for conveying a disk-shaped object on a linear path aligned on an axis from an interior of the channel to a distal port of the channel, the object translating free of rotation; and a pushrod coupled to an actuator and coupled to the channel, the pushrod having an end configured to exert a motive force on the object, and wherein the actuator is repositionable relative to the channel and along the axis.

Aspect 30 can include or use, or can optionally be combined with the subject matter of Aspect 29, to optionally include or use wherein the pushrod is configured for axial movement within a pushrod channel and wherein the pushrod channel is coupled to the deployment channel.

Aspect 31 can include or use, or can optionally be combined with the subject matter of one or any combination of Aspects 29 or 30 to optionally include or use wherein the deployment channel is coupled to a cannula having a round section.

Aspect 32 can include or use, or can optionally be combined with the subject matter of one or any combination of Aspect 31 to optionally include or use wherein the cannula includes a cutting edge.

Aspect 33 can include, or can optionally be combined with the subject matter of one or any combination of Aspects 1 through 32 to include or use, subject matter (such as an apparatus, a method, a means for performing acts, or a machine readable medium including instructions that, when performed by the machine, that can cause the machine to perform acts), such as can include or use receiving an object in a distal port of a deployment channel of a device, the deployment channel disposed along a longitudinal axis; manipulating an actuator coupled to the deployment channel and coupled to a pushrod disposed within a pushrod channel and aligned with the deployment channel, the pushrod axially repositionable between a retract position and an eject position, wherein a position of the pushrod relative to the pushrod channel is determined by the actuator; and ejecting the object from the deployment channel.

Aspect 34 can include or use, or can optionally be combined with the subject matter of Aspect 33, to optionally include or use wherein receiving the object includes encasing the object in the deployment channel.

Aspect 35 can include or use, or can optionally be combined with the subject matter of one or any combination of Aspects 33 or 34 to optionally include or use wherein manipulating the actuator includes sliding.

Aspect 36 can include or use, or can optionally be combined with the subject matter of one or any combination of Aspects 33 through 35 to optionally include or use wherein manipulating the actuator includes engaging a detent for positioning the pushrod relative to the pushrod channel.

Aspect 37 can include or use, or can optionally be combined with the subject matter of one or any combination of Aspects 33 through 36 to optionally include or use wherein ejecting the object includes moving the object along an incline aligned relative to the longitudinal axis.

Aspect 38 can include or use, or can optionally be combined with the subject matter of one or any combination of Aspects 33 through 37 to optionally include or use wherein ejecting the object includes exerting a curling force on the object.

Aspect 39 can include or use, or can optionally be combined with the subject matter of one or any combination of Aspects 33 through 38 to optionally include or use wherein ejecting the object includes exerting a thrusting force on the object.

Aspect 40 can include or use, or can optionally be combined with the subject matter of one or any combination of Aspects 33 through 39 to optionally include or use wherein ejecting the object includes translating the object.

Aspect 41 can include or use, or can optionally be combined with the subject matter of one or any combination of Aspects 33 through 40 to optionally include or use wherein ejecting the object includes rotating the object.

Aspect 42 can include or use, or can optionally be combined with the subject matter of one or any combination of Aspects 33 through 41 to optionally include or use further including passing a line coupled to the object through the distal port.

Aspect 43 can include or use, or can optionally be combined with the subject matter of one or any combination of Aspects 33 through 42 to optionally include or use further including exerting a tension force on the line.

Aspect 44 can include or use, or can optionally be combined with the subject matter of one or any combination of Aspects 33 through 43 to optionally include or use further including engaging a pin coupled to the pushrod with an aperture of the object, the pin aligned transverse to the longitudinal axis.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components, any of which can be combined variously. For example, where the description refers to pushrod 60A, it will be understood that this also refers to pushrod 60B and pushrod 60C. The drawings illustrate generally, by way of example, but not by way of limitation, various embodiments discussed in the present document.

FIGS. 1, 2, 3, and 4 illustrate views of a portion of a device for delivering an object, according to one example.

FIG. 5 illustrates a view of a portion of a device and a portion of an object, according to one example.

FIG. 6 illustrates a section view of a device for delivering an object, according to one example.

FIG. 7 illustrates a section view of a device for delivering an object, according to one example.

FIG. 8 illustrates an expanded view of a device for delivering an object, according to one example.

FIG. 9 illustrates a view of a portion of a device for delivering an object, according to one example.

FIGS. 10, 11, and 12 illustrate section views of a portion of a device for delivering an object, according to one example.

FIG. 13 illustrates a view of a portion of a device for delivering an object, according to one example.

FIG. 14 illustrates a section view of a portion of a device for delivering an object, according to one example.

FIG. 15 illustrates a tapered structure portion of a device, according to one example.

FIG. 16 illustrates a section view of a portion of a device for delivering an object, according to one example.

FIG. 17 illustrates a die structure portion of a device, according to one example.

FIG. 18 illustrates a portion of a device for delivering an object, according to one example.

FIG. 19 illustrates a portion of a pushrod and an object, according to one example.

FIG. 20 illustrates a flow chart of a method, according to one example.

FIG. 21 illustrates a flow chart of a method, according to one example.

### DETAILED DESCRIPTION

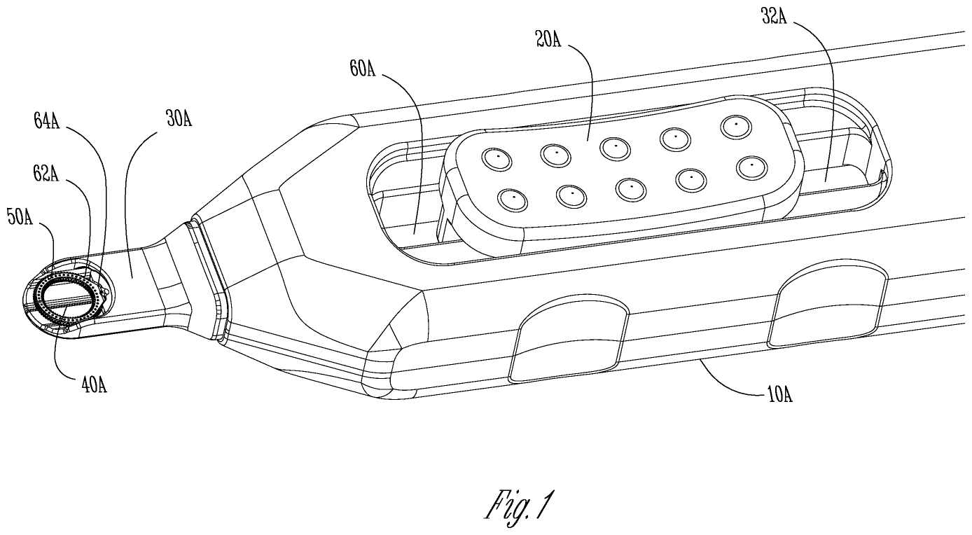

FIGS. 1, 2, 3, and 4 illustrate views of a portion of device 10A for delivering object 50A, according to one example. In these views, device 10A includes elongate deployment channel 30A, actuator 20A, and pushrod 60A.

Object 50A can include a tissue scaffold for supporting a membrane in a surgical procedure, such as macular degeneration. Object 50A can include a ring-shaped structure having a membrane (of biological tissue or non-biological material) clamped and protected between two rings of the ring-shaped structure, in the manner that a drumhead is supported by a solid ring-shape structure or a picture is supported by a frame. The membrane, having been excised from a tissue source, and the tissue may or may not have an inclination to deform or contract or shrink, curl, roll, or fold, or change shape due to variable intrinsic elastic forces. If allowed to deform or contract, cells of the membrane can be damaged. The ring-shaped structure supports the membrane in a planar configuration and protects each of the surfaces and tissue borders as the membrane is inserted through a predetermined designated tissue plane (i.e., the eye wall). An example of the present subject matter is configured to provide structural support and protection for the membrane while suspended by the ring-shaped structure. In addition, an example of the present subject matter provide a protective housing for the membrane and facilitates delivery of the membrane into the implantation site area. An example of a ring-shaped structure is noted in WO/2022/015727 TISSUE GRAFT FIXTURE.

Object 50A can be described as a disk-shaped object. According to the present subject matter, object 50A is ejected from the distal port with movement that can be described as translation. That is, object 50A changes position without rotation. The change in position refers to, at an initial time, object 50A is disposed within the interior of deployment channel and, at a later time, object 50A is clear of the deployment channel. In particular, the absence of rotation upon emergence from the distal port is specific to absence of rotation about an axis normal to the disk shape object 50A. Nevertheless, in some examples, object 50A includes an elastic structure that tends to reform after a constraint is removed. For example, object 50A can include a shape memory material and under a thermal condition, object 50A can transform from a first configuration to a second configuration. The transformation from the first configuration to the second configuration can include rotation (such as, rotation about a joint axis disposed between adjacent rings).

In the example shown at FIG. 1, object 50A is depicted at distal port 40A of deployment channel 30A. Object 50A is in a substantially planar configuration in that a top view has a circular form and an edge view has a generally rectangular horizontal form. Deployment channel 30A, in this example, has a substantially rectangular cross section configured to accommodate object 50A in the orientation shown and allow object 50A to pass through, on a linear path, without distortion or deformation. Object 50A can emerge from distal port 40A (at the end of deployment channel 30A) in line with the longitudinal axis of the deployment channel or object 50A can emerge from distal port 40A in a transverse direction via a top side aperture in deployment channel 30A.

Actuator 20A is slidably coupled to a handle portion of object 10A and in one example, is configured for finger (such as a thumb) manipulation by an operator. Actuator 20A is mechanically coupled to pushrod 60A. Pushrod 60A is disposed in pushrod channel 32A aligned substantially coaxial with deployment channel 30A. Pushrod 60A is configured to be repositionable within pushrod channel 32A. End 62A of pushrod 60A extends into deployment channel 30A. In FIG. 1, thrust surface 64A, disposed near end 62A of pushrod 60A, is in contact with object 50A.

In this view, end 62A of pushrod 60A is short of the terminus of distal port 40A. In addition, actuator 20A (coupled to pushrod 60A) is shown at a middle segment of the slide range.

FIG. 1 illustrates object 50A positioned in deployment channel 30A at a location aligned with distal port 40A.

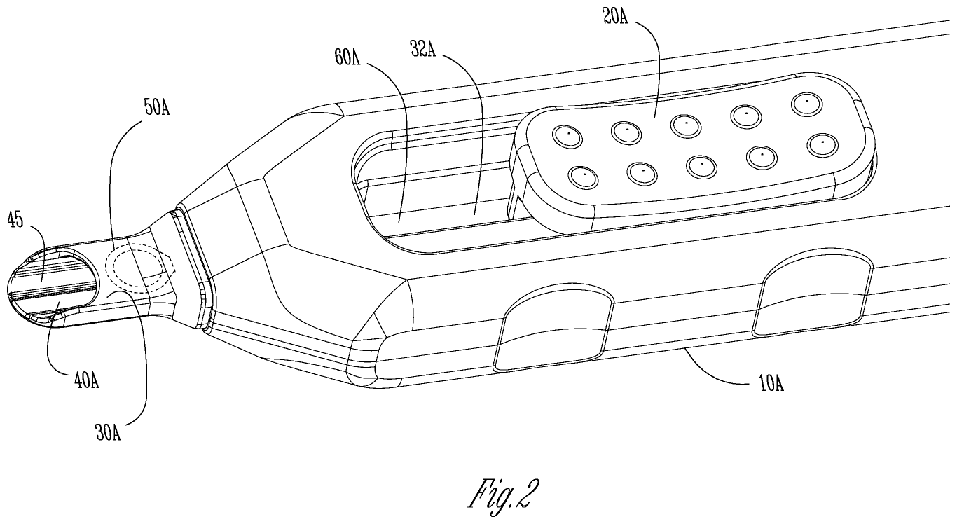

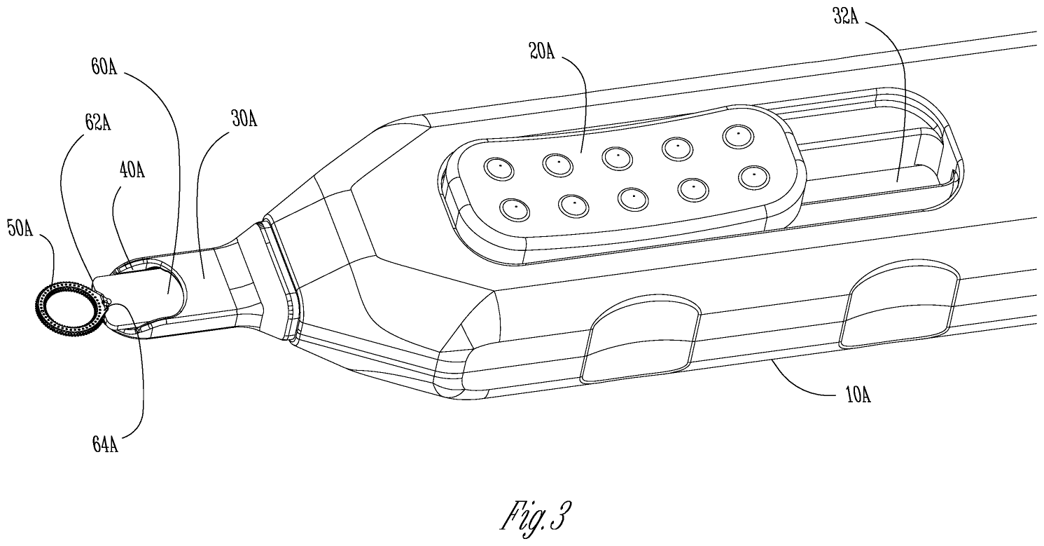

FIG. 2 illustrates object 50A within deployment channel 30A. Object 50A, when encased in deployment channel 30A as shown in the figure, is protected from damage or contaminants. Deployment channel 30A can protect object 50A during a surgical procedure, such as during insertion through a tissue plane. In this view, pushrod 60A is retracted and actuator 20A is shown at the proximal (retracted) end of the slide range. As shown in FIG. 2, device 10A can be manipulated to position distal port 40A to facilitate insertion of deployment channel 30A through an eyewall incision for implantation of object 50A, when object 50A can be ejected from device 10A, when actuator 20A is pushed toward the distal (extended) end of the slide range as schematically shown in FIG. 3.

In FIG. 3, object 50A is released from distal end 40A and ejected from deployment channel 30A. Pushrod end 62A is configured to exert a thrust force on object 50A that allows object 50A to clear distal port 40A. Actuator 20A is shown positioned at the distal end of the slide range.

Pushrod 60A can have a flat cross section, a round cross section, or a cross section of any shape. In FIGS. 1, 2, 3, and 4 for example, the section is flat and aligns well with the unfolded configuration of object 50A.

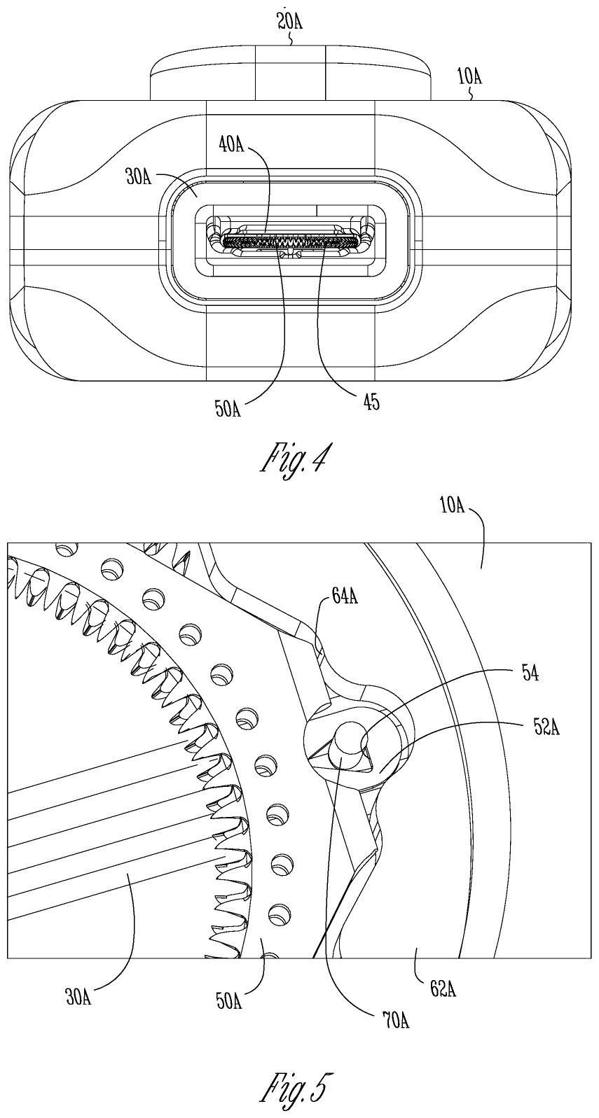

FIG. 4 illustrates an en face positional view of distal port 40A of device 10A for delivering object 50A, according to one example. The figure includes a distal edge view of object 50A at distal port 40A. This view also indicates a distal end view of deployment channel 30A, device 10A having actuator 20A.

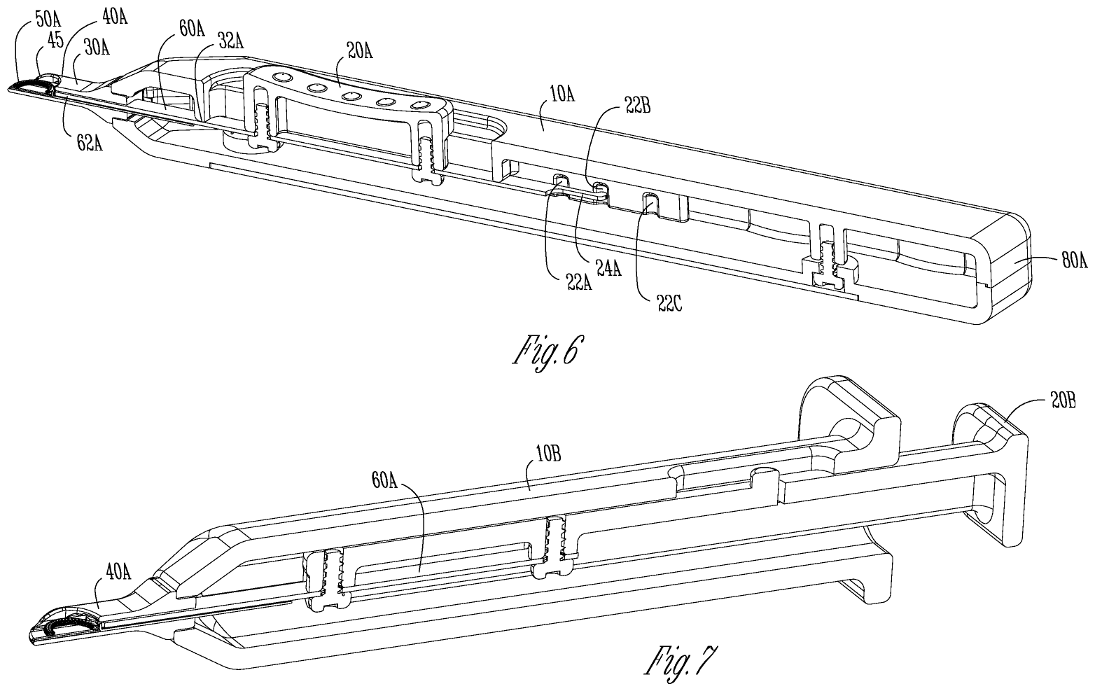

FIG. 5 illustrates a close view of a portion of device 10A and a portion of object 50A, according to one example. The figure depicts pushrod end 62A having thrust surface 64A in contact with a feature of object 50A. In addition, object 50A includes tab 52A projecting from the ring-shape form in a radial direction. Tab 52A includes aperture 54. Aperture 54 includes a substantially triangular form in the example shown. Aperture 54 is engaged by pin 70A coupled to pushrod end 62A. Pin 70A is aligned transverse to the axis and can be manipulated, with movement of pushrod end 62A, to urge object 50A to translate in an inward direction, or in an outward direction, relative to deployment channel 30A. In one example, thrust surface 64A provides a driving force to eject object 50A in a distal or outward direction.

FIG. 6 illustrates a section view of device 10A for delivering object 50A, according to one example. In this view, object 50A is shown in aperture 45 of distal port 40A. Distal port 40A is disposed at a terminus of deployment channel 30A. In this example, pushrod 60A is disposed in pushrod channel 32A and a portion extends into deployment channel 30A. Pushrod end 62A is disposed proximate object 50A. Pushrod 60A is mechanically coupled to actuator 20A. Actuator 20A, in this example, is coupled to pushrod 60A by two threaded fasteners.

In the example shown, device 10A includes a plurality of detents disposed on an internal surface of the housing. Detents 22A, 22B, and 22C correspond with selected positions of the combination of pushrod 60A and actuator 20A. Detents 22A, 22B, and 22C are engaged by elastic lever 24A coupled to pushrod 60A. Sliding action of the combination of pushrod 60A and actuator 20A, relative to pushrod channel 32A, is checked by elastic lever 24A and detents 22A, 22B, and 22C. Proximal end 80A is disposed at an end of device 10A.

A variety of mechanisms can be configured to advance a pushrod for controlling placement of object 50A. For example, a push-button mechanism, similar to that of a mechanical lead pencil, disposed at proximal end 80A can be configured to incrementally grasp and release the pushrod in a manner to control ejection of object 50A.

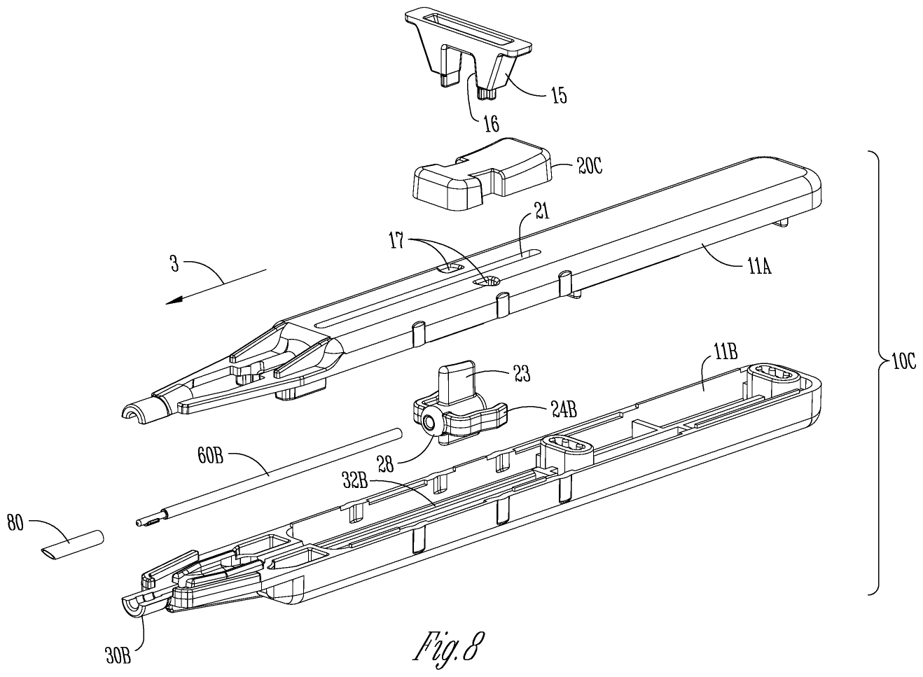

FIG. 7 illustrates a section view of device 10B for delivering an object, according to one example. In this example, actuator 20B is configured in a manner similar to a syringe plunger. In this example, thumb pressure applied to actuator 20B in an axial direction can be configured to advance pushrod 60A and eject an object from distal port 40A.

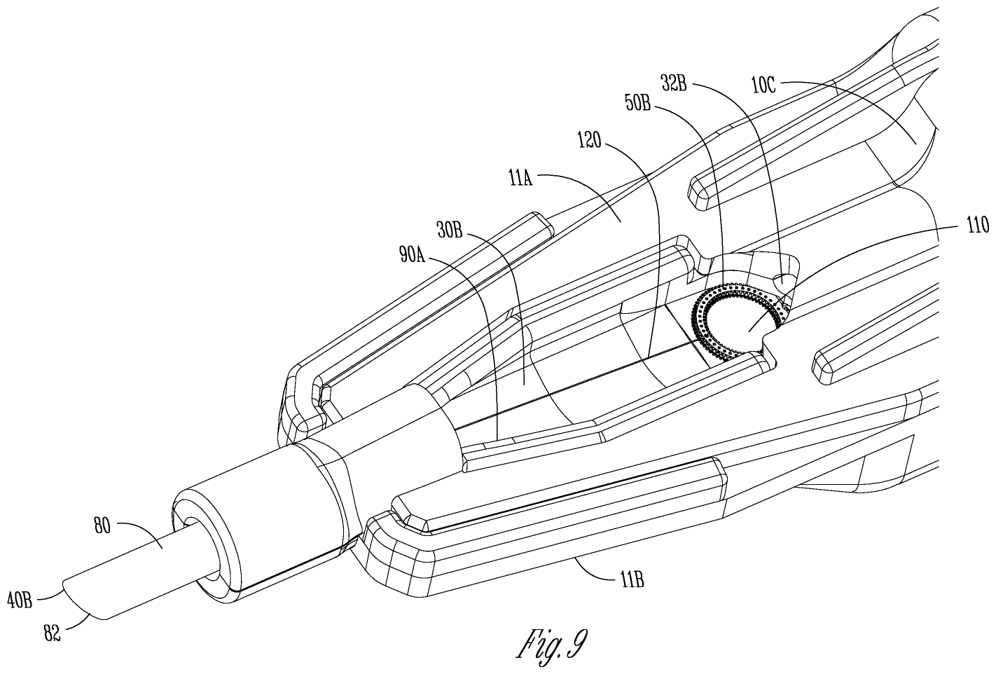

FIG. 8 illustrates an expanded view of device 10C for delivering an object, according to one example. Device 10C includes first housing segment 11A and second housing segment 11B. Pushrod channel 32B is disposed on the interior of first housing segment 11A and second housing segment 11B. Pushrod 60B is configured for axial movement within pushrod channel 32B. A proximal end of pushrod 60B is coupled to pushrod coupler 28. Pushrod coupler 28 connects pushrod 60B to actuator 20C. Actuator 20C is affixed to pushrod coupler 28 by actuator coupler 23. Actuator coupler 23 is configured to slidably engage with actuator slot 21 in first housing segment 11A. Pushrod coupler 28 includes elastic lever 24B projecting in a manner to engage with a plurality of detents on a segment of first housing segment 11A, second housing segment 11B, or both first housing segment 11A and second housing segment 11B.

Manipulation of actuator 20C causes pushrod 60B to travel in an axial direction, as indicated by arrow 3, to eject an object and to travel in a direction opposite to that of arrow 3 to retract a device. Pushrod 60B is configured to move an object within deployment channel 30B. Cannula 80 is affixed to an end of device 10C and provide a transition for carrying the object to the surgical site.

Arrow 3 can indicate an axis of movement associated with object 50A in the deployment channel 30B, an axis of movement associated with pushrod 60B, or an axis of movement associated with actuator 20C. In particular, arrow 3 can indicate an axis associated with passage of object 50A along a linear path from an interior of deployment channel 30B to distal port 40C.

The example shown in the figure also illustrates an actuator retention mechanism. In this example, the retention mechanism includes lock 15. Lock 15 has saddle 16 configured to mechanically engage with corresponding features of actuator 20C and couple with corresponding apertures 17 in first housing segment 11A. Lock 15 can provide a mechanical barrier sufficient to immobilize actuator 20C relative to first housing segment 11A and second housing segment 11B. Lock 15 can be removed from coupling with first housing segment 11A and actuator 20C with an upward force applied by hand.

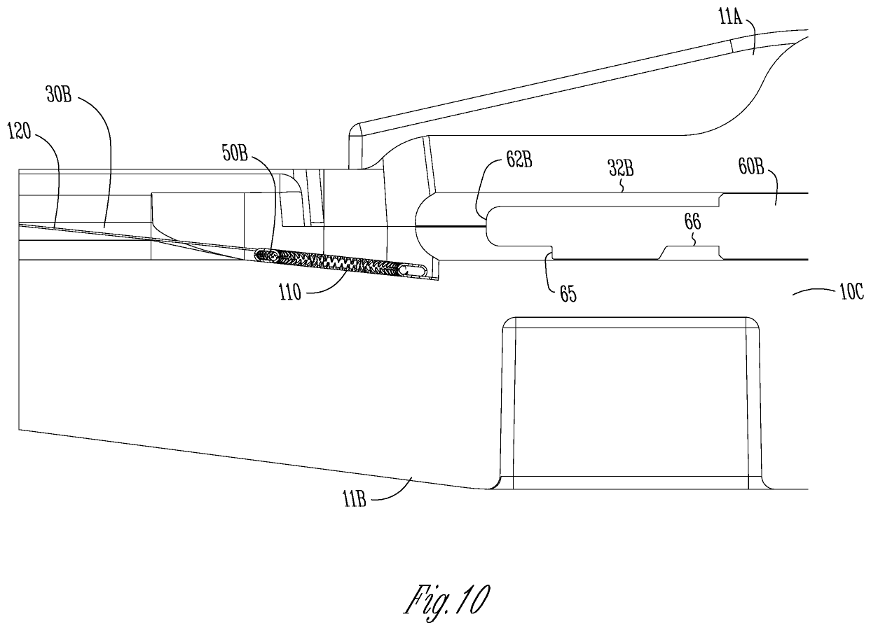

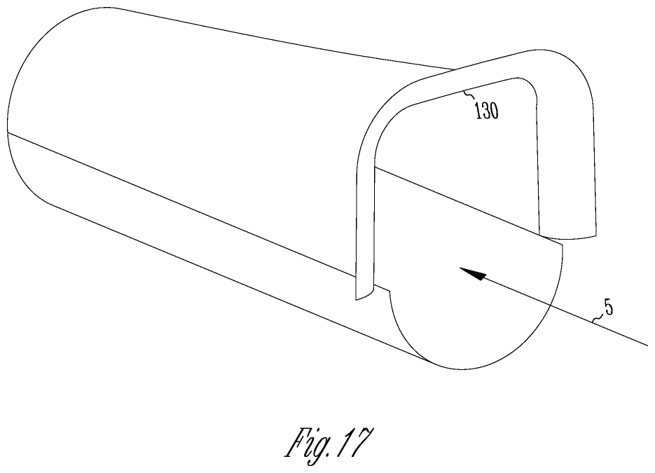

FIGS. 9, 10, 11, 12, 13, and 18 can be viewed as a time sequence by which an object is deployed. FIG. 9 can be viewed as an initial condition in which the object is positioned for delivery and FIG. 18 can be viewed as a time when the object is deployed at the surgical site.

FIG. 9 illustrates a view of a portion of device 10C for delivering object 50B, according to one example. In this example, object 50C is carried in a planar configuration while in an internal cavity of device 10C, and with passage through deployment channel 30B, object 50C is formed into a shape that can pass through a smaller incision at a surgical site.

Device 10C includes first housing segment 11A and second housing segment 11B. In the figure, pushrod channel 32B has an aperture near a storage position of object 50B. Object 50B is in a planar configuration. Angular walls adjacent the storage position further facilitate retention of object 50B.

Object 50B is ejected from device 10C via passage through deployment channel 30B. In the example shown, two mechanisms can translate object 50B. Line 120 can be affixed to object 50B by a suture, a bond, or other means, and routed through deployment channel 30B. A tensile force applied manually to line 120 can carry object 50B to eject through distal port 40B. Line 120 can include a thread, a fiber, or a suture.

In addition, a pushrod moved axially within pushrod channel 32B can exert a thrust force to eject object 50B.

Distal port 40B includes an angle cut end 82. Angle cut end 82 provides a directional deployment of object 50B as it exits the distal port 40B. In addition, in the event that object 50B includes shape memory materials, angle cut end 82 allows object 50B to slowly reform into a planar configuration. Cannula 80 terminates at distal port 40B. Cannula 80 has a circular section and provides a configuration suitable for a surgical procedure as described herein. In one example, angle cut end 82 includes a cutting edge suitable for cutting and penetrating tissue. In this example, angle cut end 82 is sharpened to an acute angle suitable for a cutting edge.

Object 50B can be reformed from an initial, planar configuration, in a controlled manner by both the deployment of pulling force from line 120 and pushing force (exerted by pushrod 60B) as shown, to provide operator-controlled deployment, suitable for implantation, and passage of object 50B through the wall of the eye or other organ as part of a surgical procedure. As a function of passage through deployment channel 30B, one example alters object 50B from a planar configuration to a folded or rolled configuration. Forming can be provided by conical or similarly shaped surfaces through which object 50B passes in the course of deployment. For example, the conical or tapered wall 90A is disposed adjacent the pathway by which object 50B emerges. Tapered wall 90A can be configured to impart a bend or curve to a portion of object 50B.

In one example, object 50B includes a shape memory material. At a selected temperature, object 50B can be configured for elasticity and become malleable for passage through the tapered wall 90A, while at another temperature, object 50B will reform to a predetermined shape. For example, object 50B can be placed as shown in FIG. 9 while in a flat, or planar configuration, and upon ejection from distal port 40B, object 50B can be in a coiled or rolled form, and upon reaching body temperature, object 50B returns to a planar configuration.

In the example shown, object 50B is supported by surface 110. Surface 110 is pitched at an incline in a manner whereby the object portion nearest distal port 40B is elevated relative to the object portion farthest from distal port 40B.

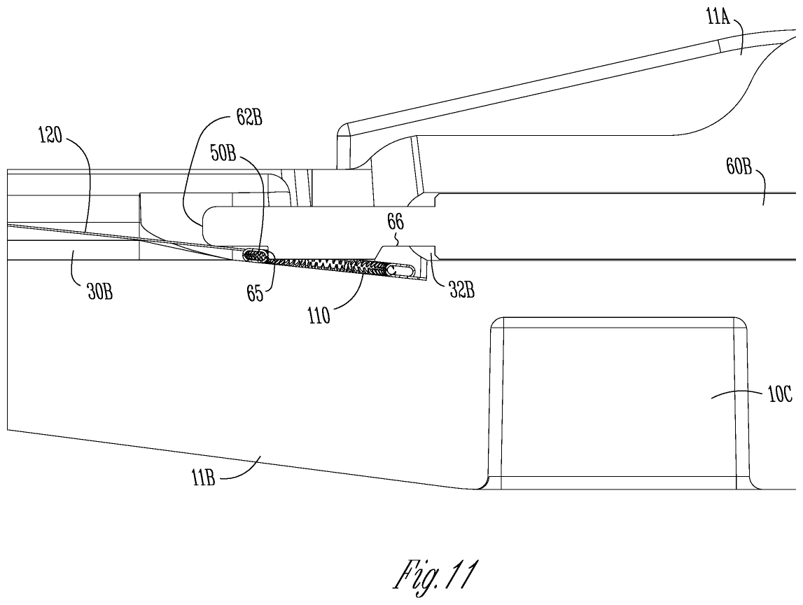

FIG. 10 illustrates a section view of a portion of device 10C for delivering object 50B, according to one example. Device 10C includes first housing segment 11A and second housing segment 11B. In the example shown, pushrod channel 32B, disposed between segment 11A and segment 11B, carries pushrod 60B.

Pushrod 60B includes end 62B, shoulder 65, and relief 66. End 62B is configured to pass clear of object 50B when pushrod 60B is translated in a direction to eject object 50B. Shoulder 65 is configured to exert a thrust force on a portion of object 50B. Shoulder 65 is configured to pass clear of a first portion of object 50B and engage a second portion of object 50B. Object 50B, in the example shown, is disposed on inclined surface 110. Relief 66 is configured to receive the second portion of object 50B after displacement and partial rotation of object 50B in the course of passage through deployment channel 30B. Line 120 is shown coupled to object 50B.

FIG. 11 illustrates a section view of a portion of device 10C for delivering object 50B, according to one example. In this view, pushrod 60B is positioned within pushrod channel 32B and having shoulder 65 in contact with an interior surface (here, an inner diameter) of object 50B. Object 50B is shown disposed on inclined surface 110 and clear of relief 66. Line 120 is affixed to object 50B.

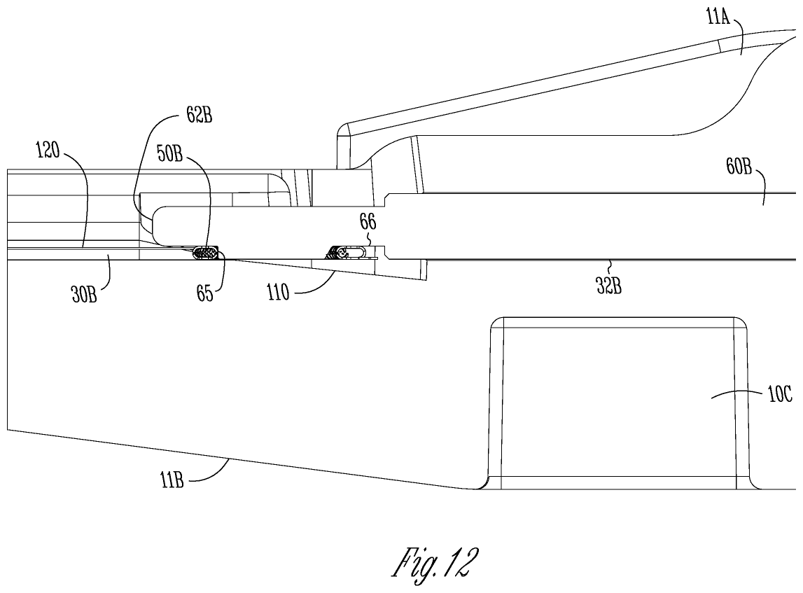

FIG. 12 illustrates a section view of a portion of device 10C for delivering object 50B, according to one example. In this view, pushrod 60B has advanced within pushrod channel 32B. Shoulder 65 remains in contact with an interior surface of object 50B. Object 50B is shown above, and not in contact with, inclined surface 110. In this view, a plane of object 50B is substantially parallel with an axis of deployment channel 30B. Object 50B is engaged with relief 66. Line 120 remains affixed to object 50B. As between the view in FIG. 11 and the view in FIG. 12, object 50B has undergone rotation from a position on inclined surface 110 to a position in the deployment channel 30B.

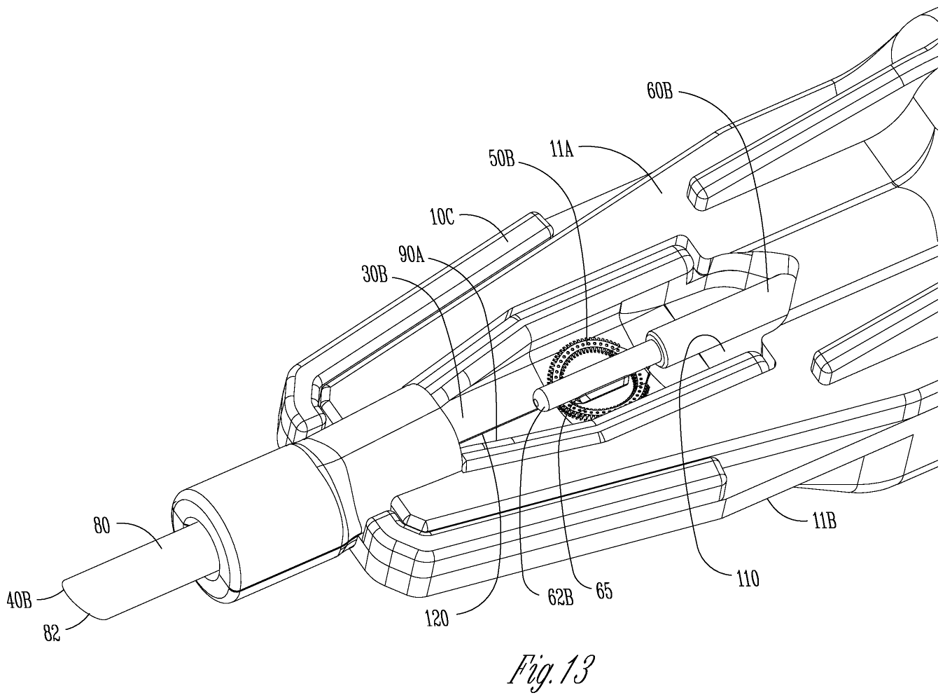

FIG. 13 illustrates a perspective view of a portion of device 10C for delivering object 50B, according to one example. In this view, pushrod 60B has further advanced within pushrod channel 32B. Shoulder 65 remains in contact with an interior surface of object 50B. Object 50B is shown to have cleared inclined surface 110. Object 50B remains substantially parallel with an axis of deployment channel 30B. Object 50B remains engaged with relief 66. Line 120 remains affixed to object 50B. Object 50B remains clear of tapered wall segment 90A, and as such, retains a substantially planar form. As between the view in FIG. 12 and the view in FIG. 13, object 50B has undergone translation from a first position near inclined surface 110 to a second position along a length of deployment channel 30B.

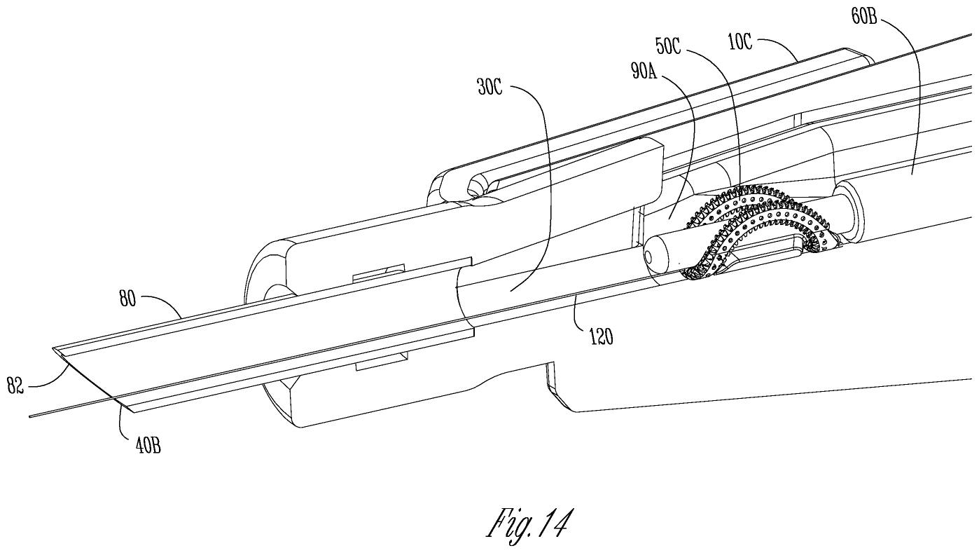

FIG. 14 illustrates a perspective view of a portion of device 10C for delivering object 50C, according to one example. In this view, pushrod 60B has further advanced within the pushrod channel, and a portion of pushrod 60B is adjacent the conical or tapered wall segment 90A. Object 50C is shown to have a folded shape. Object 50C has taken the indicated shape after having passed through tapered wall segment 90A of deployment channel 30C. Object 50C remains engaged with a shoulder portion and a relief portion of pushrod 60B. Line 120 remains affixed to object 50B.

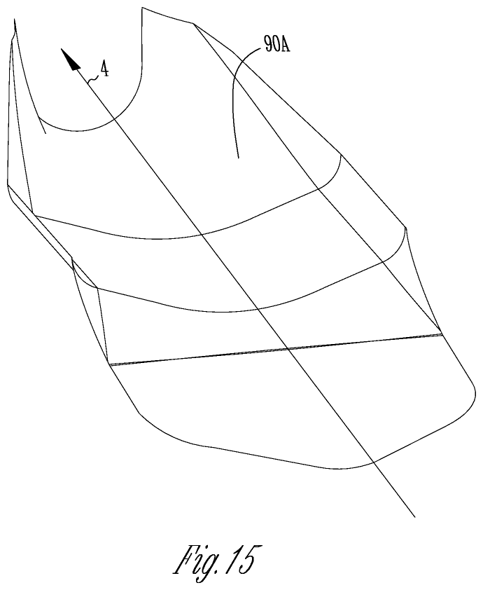

FIG. 15 illustrates a tapered structure portion of a device, according to one example. In the example shown tapered wall segment 90A is provided on an interior surface, and as object 50C passes in the direction indicated by arrow 4, portions of object 50C are deflected in an upward direction to form a ‘u-shape’ profile.

FIG. 16 illustrates a perspective view of a portion of device 10C for delivering object 50C, according to one example. In this view, pushrod 60B has further advanced within the pushrod channel. Object 50C is shown to have a rolled or coiled shape. Object 50C has taken the indicated shape after having passed through tapered wall segment and through curling die 130A. Curling die 130A includes a contoured surface of deployment channel 30C. Curling die 130A, in the example shown, brings together the upper sides of object 50C. Object 50C remains engaged with a shoulder portion and a relief portion of pushrod 60B. Line 120 remains affixed to object 50B.

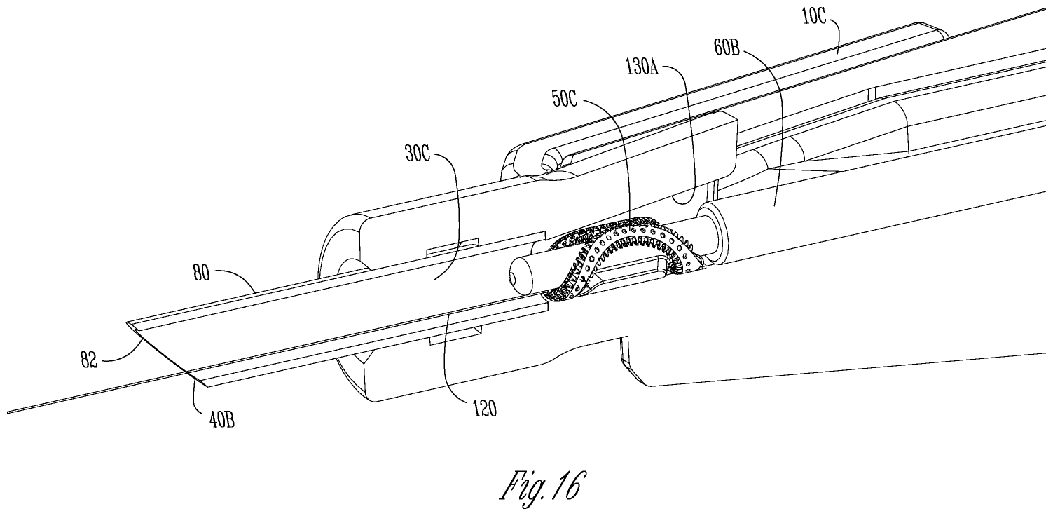

FIG. 17 illustrates a portion of a device, according to one example. In the example shown, curling die 130B is provided on an interior surface, and as object 50C passes in the direction indicated by arrow 5, portions of object 50C are deflected in a direction to curl object 50C into a tubular profile.

FIG. 18 illustrates a perspective view of a portion of device 10C for delivering object 50D, according to one example. Device 10C includes first housing segment 11A and second housing segment 11B. In this view, actuator 20C is displaced in a distal, or forward, position of actuator slot 21, and as such, the pushrod has ejected object 50D clear of angular cut 82 at distal port 40B. Line 120 remains affixed to object 50D. Object 50D is shown to have a rolled or coiled shape.

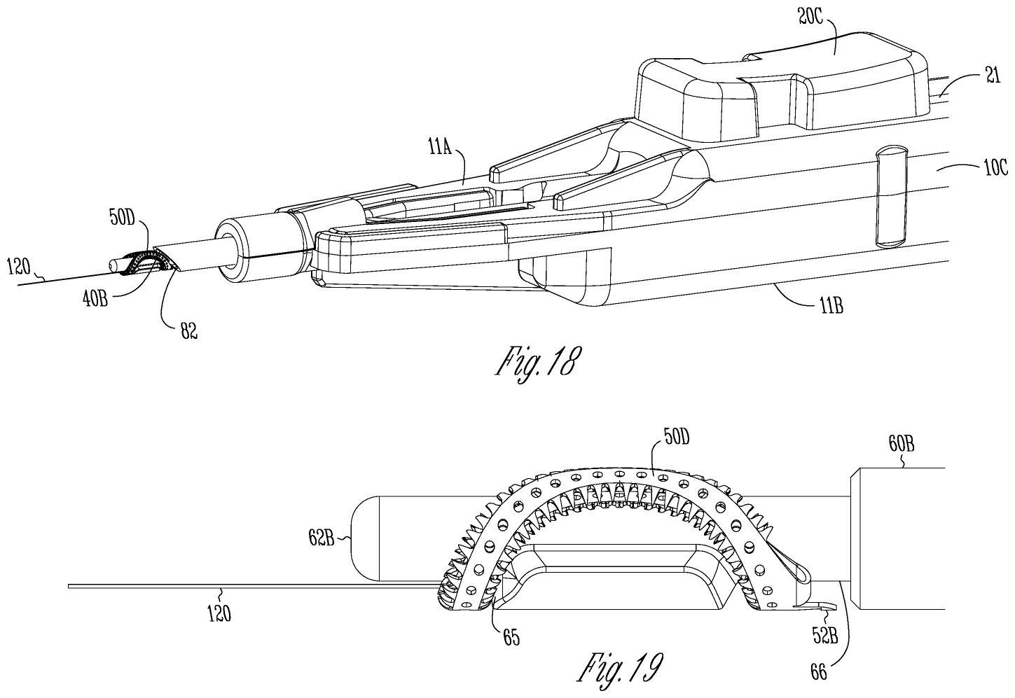

FIG. 19 illustrates a portion of pushrod 60B and object 50D, according to one example. Pushrod 60B has end 62B which extends beyond the major diameter of object 50D. Shoulder 65 of pushrod 60B is engaged with an interior diameter of object 50D and relief 66 provides clearance for object 50D. Object 50D is shown to partially wrap about the diameter of pushrod 60B. Object tab 52B projects from object 50B and provides a structure by which object 50B can be grasped and manipulated. Line 120 is affixed to object 50D and emerges from below pushrod 60B.

Object 50C can pass through the deployment channel 30C under a force exerted by the pushrod 60B. In one example, pushrod 60B contacts object 50C on a thrust surface that can include an end portion of pushrod 60B. The end portion of the pushrod 60B can include a face end of the pushrod, a surface of a transverse pin (such as pin 70A) disposed near the face end of the pushrod, and a shoulder (such as shoulder 65) of the pushrod 60B that is set back from the face end. The force exerted on object 50C, by pushrod 60B, can be described as a thrust force in that it aligns with a direction of deployment as defined by the deployment channel 30C. The force exerted on object 50C, by pushrod 60B, can also be described as a motive force in that the force results in movement of object 50C relative to the deployment channel 30C. The force exerted on object 50C is configured to propel the object through deployment channel 30C.

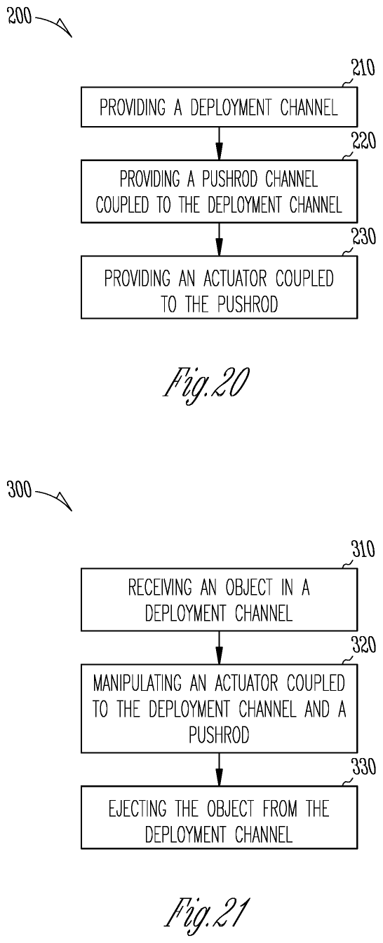

FIG. 20 illustrates a flow chart of method 200, according to one example. Method 200 relates to a manufacturing process by which a device can be produced. At 210, method 200 includes providing a deployment channel. The deployment channel can be fabricated by molding, casting, or by printing using additive manufacturing machinery. The deployment channel can be fabricated of a polymer, a metal, or other substantially rigid material.

The deployment channel can be configured to deliver a planar device, such as object 50A, in an undeformed planform. As such, the deployment channel can have a substantially rectangular section. The rectangular section can be sized and configured to deploy, an object suited for supporting a membrane in planar form. In one example, the deployment channel can be sized and configured to receive an object suited for supporting a membrane in a planar form.

The deployment channel can be configured to deliver a planar device, such as object 50A, in a folded planform. As such, the deployment channel can have a first section that receives a substantially planar object and have a second section that deploys the object in a folded form. A length between the first section and the second section can include contoured or angular wall sections which deform the object (initially relatively flexible and relatively flat) with progression through the deployment channel. In some examples, the folded form of the object upon emergence from the deployment channel can resemble a ‘u-shape’ or a chevron in that a first portion of the object undergoes bending and other portions of the object remain undeformed. In some examples, the folded form of the object upon emergence from the deployment channel can be tubular or coiled in that the object undergoes bending or shaping to form a substantially circular section.

At 220, method 200 includes providing a pushrod channel coupled to the deployment channel. The pushrod channel can be configured to have rectangular section, a round section, or other form. The pushrod channel is sized and configured to receive a pushrod and allow axial movement therebetween. The pushrod and pushrod channel can be fabricated of a polymer, a metal, or other substantially rigid material. In one example, the pushrod channel and the deployment channel are coaxial.

At 230, method 200 includes providing an actuator coupled to the pushrod. The actuator can include a slidably coupled member having a high friction surface on a first segment and having a low friction surface on a second segment. The high friction surface is configured to receive an exerted thrust force from a finger and the low friction surface is configured for sliding relative to a housing segment of the pushrod channel.

Method 200 can include variations and can include additional proceedings. For example, the pushrod and the actuator can be of unitary structure and integrated into a single component. In other examples, method 200 can include procedures for fabricating a structure as described herein.

FIG. 21 illustrates a flow chart of method 300, according to one example. Method 300 relates to a process by which a device can be operated. At 310, method 300 includes receiving an object in a deployment channel. In some examples, this includes engaging a pin with an aperture (or socket) of the object. In some examples, this includes manually positioning the object in a deployment channel using a forceps or other instrument.

At 320, method 300 includes manipulating an actuator coupled to the deployment channel and a pushrod. The actuator can be configured to receive a thrust force applied by a digit of an operator. The actuator can be coupled to the pushrod disposed in a pushrod channel. The coupling can be rigid or flexible. Manipulation of the actuator causes the pushrod to translate and exert a force on the object disposed in a deployment channel in communication with the pushrod channel.

At 330, method 300 includes ejecting the object from the deployment channel. The object is ejected by a translational force applied by an axially displaced pushrod. The object can emerge from the deployment channel in a planar form or emerge in a folded form. In some example, the object undergoes deformation during the passage in the deployment channel.

Method 300 can include variations and can include additional proceedings. For example, manipulation of the actuator can also draw the object into the deployment channel. A transverse pin of the pushrod can engage with the object and exert a translation force to bring the object into the deployment channel. In other examples, method 300 can include procedures for folding or bending the object during passage through the deployment channel.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A device comprising:
a deployment channel having a distal port and a proximal end disposed along a longitudinal axis; and
an actuator coupled to the deployment channel and coupled to a pushrod disposed within a pushrod channel and aligned with the deployment channel, the pushrod axially repositionable between a retract position and an eject position, wherein a position of the pushrod relative to the pushrod channel is determined by the actuator.

2. The device of claim 1 wherein the pushrod includes an engagement pin aligned transverse to the deployment channel.

3. The device of claim 1 wherein the deployment channel is tubular.

4. The device of claim 1 wherein a cross section of the deployment channel is substantially rectangular.

5. The device of claim 1 wherein the distal port includes an elongate aperture.

6. The device of claim 1 wherein the actuator is slidably coupled to the deployment channel.

7. The device of claim 1 further including a detent for positioning the pushrod relative to the pushrod channel.

8. The device of claim 1 wherein the actuator includes a syringe plunger.

9. The device of claim 1 wherein the deployment channel includes a surface inclined relative to the longitudinal axis.

10. The device of claim 9 wherein the surface is disposed proximate the distal port.

11. The device of claim 1 wherein the deployment channel includes a curl forming die.

12. The device of claim 1 wherein the pushrod includes a round section.

13. The device of claim 1 wherein the pushrod includes a shoulder at a selected axial position.

14. The device of claim 13 wherein the shoulder is configured to exert a thrust force aligned with the longitudinal axis.

15. The device of claim 13 wherein the deployment channel is configured to clear an end of the pushrod and wherein the shoulder is configured to exert a thrust.

16. The device of claim 15 wherein the pushrod includes a relief disposed proximate the end.

17. The device of claim 1 wherein the distal port includes a cannula.

18. The device of claim 17 wherein the cannula includes an angle cut end.

19. The device of claim 17 wherein the cannula includes a cutting edge.

20. The device of claim 1 further including a lock configured to immobilize the pushrod relative to the pushrod channel.

21. The device of claim 20 wherein the lock includes an actuator saddle.

22. The device of claim 1 wherein the deployment channel has a tapered wall disposed proximate the distal port.

23. The device of claim 22 wherein the tapered wall includes a substantially circular cross section.

24. A device comprising:
a channel having an interior configured to receive a disk-shaped object and having a distal port for deploying the object;
a pushrod configured for movement between a first axial position distal to the channel and a second axial position proximate to the channel, wherein an end of the pushrod is configured to exert a thrust force on the object and propel the object along an axis between the interior and the distal port; and
an actuator coupled to the pushrod.

25. The device of claim 24 wherein the channel includes a wall at an angle relative to the axis.

26. The device of claim 24 wherein the interior is configured to deform the object with passage along the axis.

27. The device of claim 24 wherein the distal port includes a cannula.

28. The device of claim 27 wherein the cannula includes a cutting edge.

29. A device comprising:
a deployment channel configured for conveying a disk-shaped object on a linear path aligned on an axis from an interior of the channel to a distal port of the channel, the object translating free of rotation; and
a pushrod coupled to an actuator and coupled to the channel, the pushrod having an end configured to exert a motive force on the object, and wherein the actuator is repositionable relative to the channel and along the axis.

30. The device of claim 29 wherein the pushrod is configured for axial movement within a pushrod channel and wherein the pushrod channel is coupled to the deployment channel.

31. The device of claim 29 wherein the deployment channel is coupled to a cannula having a round section.

32. The device of claim 31 wherein the cannula includes a cutting edge.
